(19) United States
(12) Patent Application Publication
(10) Pub. No.: US 2024/0398615 A1
(43) Pub. Date: Dec. 5, 2024

(54) PORTABLE ROBOTIC OCCULAR DRUG DELIVERY DEVICE

(71) Applicant: Alcon Inc., Fribourg (CH)

(72) Inventors: Nanhong LOU, Southlake, TX (US); Patrick TERRY, Goleta, CA (US); Lance NOLLER, Lake Forest, CA (US); Mark Andrew ZIELKE, Lake Forest, CA (US); Lingfeng YU, Lake Forest, CA (US)

(21) Appl. No.: 18/677,634

(22) Filed: May 29, 2024

Related U.S. Application Data
(60) Provisional application No. 63/505,496, filed on Jun. 1, 2023.

(51) Int. Cl. A61F 9/00

(52) U.S. Cl. CPC A61F 9/0008

(57) ABSTRACT

A drug delivery device includes a drug delivery assembly configured to receive a portion of a head of a patient and mounted to a helmet or support. The drug delivery assembly includes one or more imaging devices configured to have an eye of the patient in a field of view thereof, an injection assembly configured to receive a drug to be injected, and a staging assembly including one or more actuators configured to position the injection assembly relative to the eye of the patient. A controller receives one or more images from the one or more imaging devices; detects a location of a limbus of the eye of the patient in the one or more images; and activate the one or more actuators to drive a needle into a placement location on the eye of the patient and dispense a drug into the eye through the needle.

### CROSS-REFERENCE TO RELATED APPLICATIONS

This application claims priority to and benefit of U.S. Provisional Patent Application No. 63/505,496, filed Jun. 1, 2023, which is hereby assigned to the assignee hereof and hereby expressly incorporated by reference in its entirety as if fully set forth below and for all applicable purposes.

### INTRODUCTION

The present disclosure relates generally to instruments used for providing intravitreal injections.

Light received by the eye is focused by the cornea and lens of the eye onto the retina at the back of the eye, which includes the light sensitive cells. The interior of the eye between the lens and the retina is filled with a transparent gel known as the vitreous. Many conditions of the retina are treated by intravitreal injections in which medication is injected into the vitreous. Such conditions include age-related macular degeneration, retinal vein occlusion, diabetic macular edema, diabetic retinopathy, and others. Once diagnosed with a condition requiring intravitreal injections, a patient may continue to require injections periodically.

It would be an advancement in the art to facilitate the administration of intravitreal injections.

### SUMMARY

In certain embodiments, a drug delivery device includes a drug delivery assembly configured to receive a portion of a head of a patient. The drug delivery assembly includes one or more imaging devices configured to have an eye of the patient in a field of view thereof and an injection assembly configured to receive a drug to be injected into the eye of the patient. The drug delivery assembly further includes a staging assembly including one or more actuators and configured to position the injection assembly relative to the eye of the patient. A controller is coupled to the one or more imaging devices and the staging assembly and is configured to receive one or more images from the one or more imaging devices. The controller is configured to detect a location of a limbus of the eye of the patient in the one or more images. The controller is configured to activate the one or more actuators to drive a needle mounted to the injection assembly into a placement location on the eye of the patient according to the location of the limbus and dispense a drug into the eye through the needle.

In certain embodiments, a method for drug delivery includes: positioning a portion of a head of a patient in a drug delivery assembly comprising one or more imaging devices configured to have an eye of the patient in a field of view thereof, an injection assembly configured to receive a drug to be injected into the eye of the patient, and a staging assembly including one or more actuators and configured to position the injection assembly relative to the eye of the patient; receiving, by a controller, one or more images from the one or more imaging devices; detecting, by the controller, a location of a limbus of the eye of the patient in the one or more images; and activating, by the controller, the one or more actuators to drive a needle mounted to the injection assembly into a placement location on the eye of the patient according to the location of the limbus.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1A is a side view of a portable robotic intravitreal drug delivery device in accordance with certain embodiments.

FIG. 1B is a top view of the device of FIG. 1A.

FIG. 2A is a side view of an alternative embodiment of a portable robotic intravitreal drug delivery device in accordance with certain embodiments.

FIG. 2B is a top view of the device of FIG. 2A.

FIG. 3 is a schematic diagram of components for implementing a robotic intravitreal drug delivery device in accordance with certain embodiments.

FIGS. 4A and 4B illustrate actuators for an injection assembly in accordance with certain embodiments.

FIGS. 5A to 5D illustrate example injection assemblies in accordance with certain embodiments.

FIG. 6 is a schematic block diagram of electronic components of a portable robotic intravitreal drug delivery device in accordance with certain embodiments.

FIG. 7 is a process flow diagram of a method for preparing for drug delivery using a portable robotic intravitreal drug delivery device in accordance with certain embodiments.

FIG. 8 is a process flow diagram of a method for administering an intravitreal injection using a portable robotic intravitreal drug delivery device in accordance with certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

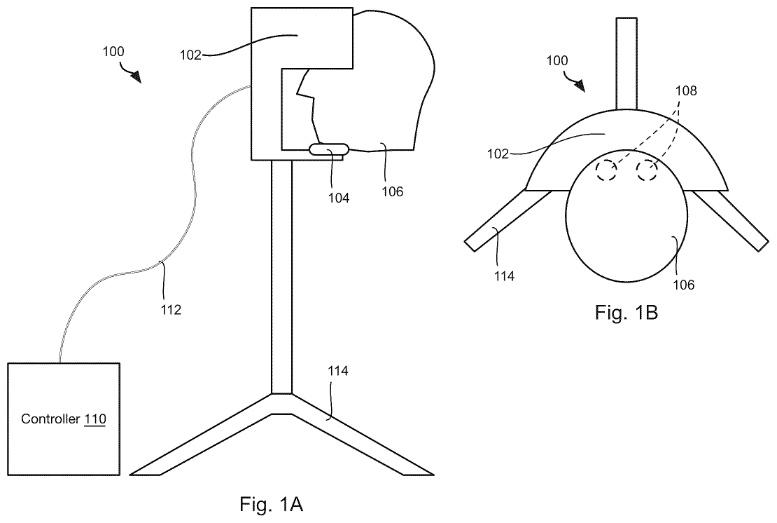

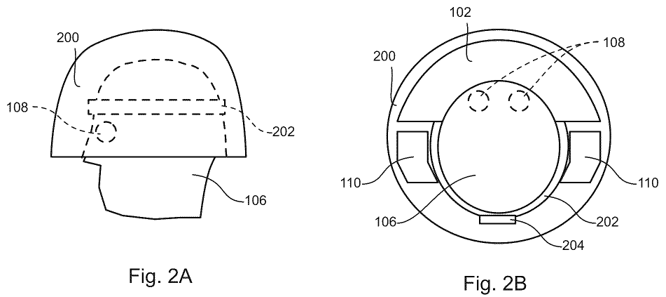

FIG. 1A illustrates an example portable robotic intravitreal drug delivery device 100 (hereinafter “drug delivery device 100”). The drug delivery device 100 includes a drug delivery assembly 102 coupled to a chin rest 104. The drug delivery assembly 102 receives a portion of the head 106 of a patient with the chin of the patient resting on the chin rest 104. The drug delivery assembly 102 may include some or all of a forehead rest, cheek rests, temporal rests, or rests for engaging other portions of the head 106 of the patient. The drug delivery assembly 102 may include structures for clamping or otherwise retaining the head 106 of the patient, such as a clamp for pressing pads against temporal regions of the patient's head 106, a headband encircling the patient's head 106 and fastened to the drug delivery assembly 102, or other structures. Alternatively, the patient may be relied upon to press the patient's head against the drug delivery assembly 102 and chin rest 104 with sufficient stability for the administration of an intravitreal injection as described below. The drug delivery assembly 102 is positioned around and over the eyes 108 of the patient.

The drug delivery assembly 102 may be housed separately from a controller 110 and connected to the controller 110 by means of a cable 112 or wireless connection in order to reduce the weight of the drug delivery assembly 102. The controller 110 includes logic (e.g., a computing device) for controlling actuators and other components of the drug delivery assembly 102. The controller 110 may include a power supply such as a rechargeable battery or adapter for connecting to an electrical outlet.

The drug delivery assembly 102 and chin rest 104 may be mounted to a support 114. The support 114 may be configured to rest on a floor, countertop, or other stable support surface with the drug delivery assembly 102 and chin rest 104 vertically positioned to receive the head 106 of a patient that is seated adjacent to the support 114. The support 114 may, for example, be embodied as the illustrated tripod that may be collapsible to facilitate easy transportation. The support 114, such as the illustrated tripod, may have integrated or detachable weights, stabilizing feet, or other features to provide stability during use. The support 114 may be adjustable vertically to facilitate positioning the drug delivery assembly 102 and chin rest 104 at a height that is comfortable to the patient. The chin rest 104 may likewise be adjustable.

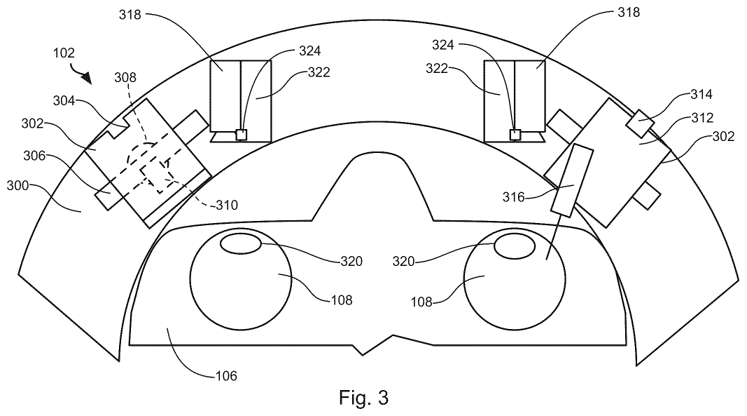

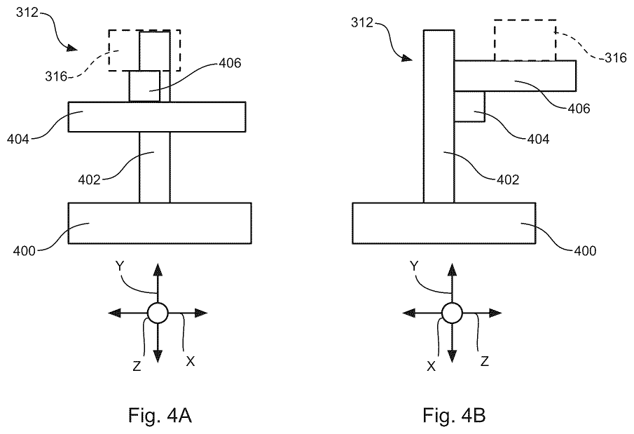

Referring to FIGS. 2A and 2B, in an alternative implementation, the drug delivery assembly 102 is mounted to a helmet 200 that is worn on the head 106 and over the eyes 108 of the patient. The controller 110 may be mounted within the helmet 200 or may be housed separately and connected by a cable 112 as for the embodiment of FIGS. 1A and 1B. The helmet 200 may include a headband 202 and tensioner 204. A patient or other operator may tension the headband 202 using the tensioner 204 to snugly hold the patient's head 106 within the helmet 200 and urge a forehead of the patient's head 106 against the drug delivery assembly 102 in order to reduce movement of the patient's head 106 relative to the helmet 200 during drug delivery. The helmet 200 may provide the advantage of leaving the patient's eyes 108 without significant light thereby reducing the visibility of the needle of the drug delivery assembly 102 and thereby reducing flinching and other movement.

Although the embodiments of FIGS. 1A, 1B, 2A, and 2B, show the patient's head 106 oriented as when seated or standing, other embodiments may be used with the patient reclined, such as reclined and facing upward.

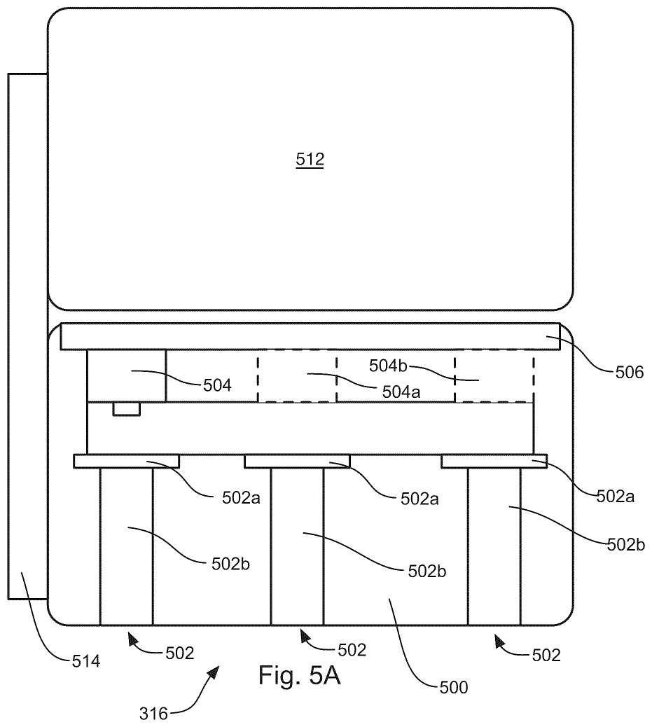

Referring to FIG. 3, the drug delivery assembly 102 includes a frame 300. The frame 300 may be embodied as a track or rail along which components may be fastened at various positions. The frame 300 may include one or more mounting structures 302 mounted thereto. The mounting structure 302 includes one or more registration features 304 enabling rigid and repeatable mounting to the mounting structure 302. The mounting structures 302 may have one or more degrees of adjustment including some or all of: (a) being securable to the frame 300 at a range of possible locations, (b) a translational adjustment mechanism 306 enabling translational adjustment of each mounting structure 302 relative to a point of attachment to the frame 300, and (c) a rotational adjustment mechanism 308 enabling rotational adjustment of the mounting structure 302 relative to a point of attachment to the frame 300. One or more locking mechanisms 310 may be used to lock the mounting structure 302 relative to the frame 300 and prevent further adjustment along the frame 300 or using the translational adjustment mechanism 306 and rotational adjustment mechanism 308. In some methods of use, the drug delivery assembly 102 is set up for an individual patient and may even be sent home with the patient. The mounting structures 302 may therefore be set up by a professional operator and then locked in place using locking mechanisms 310 in order to prevent further adjustment. The translational adjustment mechanism 306 and rotational adjustment mechanism 308 may facilitate manual adjustment by a professional operator or may include electronic actuators or interfaces to be controlled by electronic actuators. In other embodiments, the patient may adjust the positions of the mounting structure 302 to ensure the position of the patient's head 106 and eye 108 are aligned with a needle mounted to the mounting structure 302 as discussed below. For example, the controller 110 may provide guidance in the form of verbal or visible instructions based on the output of one or more cameras having the patient's eye 108 in the field of view thereof, such as the one or more cameras 318 discussed below. The customer may adjust the mounting structure manually or by controlling one or both of the translational and rotational adjustment mechanisms 306, 308 according to the guidance.

In some embodiments, a staging assembly 312 is securable to each mounting structure 302. The same staging assembly 312 may therefore be used to treat both eyes 108 of a patient. The staging assembly 312 engages the registration features 304 of the mounting structures 302 in order to hold the staging assembly 312 rigidly with respect to the mounting structure 302 and to ensure precise (e.g., within 0.01 millimeter or within 1 micron) positioning of the staging assembly 312 with respect to the mounting structure 302. The staging assembly 312 may fasten to each structure using a latch 314 that may be engaged and released by a patient without the use of tools.

In some methods of use, the patient does not need to disengage the patient's head 106 from the drug delivery assembly 102 between treatments to both the patient's eyes 108. Accordingly, there may be two staging assemblies 312 or movement of the staging assembly 312 from one mounting structure 302 to the other may be automatic, e.g., manually or actuated sliding along a track formed on or by the frame 300.

The staging assembly 312 includes one or more actuators that control the position of an injection assembly 316 mounted to the staging assembly 312. The staging assembly 312 attempts to compensate for variation in positioning of the patient's head 106 relative to the frame 300, misalignment of the patient's eyes 108, or other potential causes of variation relative to the positioning of the patient's head 106 and eyes 108 when the mounting structures 302 were configured. In some embodiments, the controller 110 performs an alignment check to facilitate alignment of the eye 108 of the patient relative to the staging assembly 312. For example, using eye tracking with images from the camera 318, the controller 110 may detect a degree of misalignment, particularly misalignment that is outside of the range of motion of the staging assembly 312, and provide feedback to the patient to correct the misalignment. The feedback may be in the form of instructions displayed on a screen, such as a screen implementing the fixation target 324. For example, the patient may be shown a box and a target corresponding to the current position of the patient's eye 108. The patient may be instructed to move the patient's head 106 relative to the drug delivery assembly 102 until the target is positioned within the box while the controller updates the position of the target based on the current position of the patient's eye 108. The patient may then clamp the patient's head 106 relative to the drug delivery assembly 102.

The amount of variation may be small such that the range of motion of the staging assembly 312 may likewise be small, such as less than 1 centimeter, less than 5 millimeters, or less than 2 millimeters alone one or more translational directions or less than 10 degrees, less than 5 degrees, or less than 2 degrees in one or more rotational directions.

Adjustment of the staging assembly 312 may be performed using feedback from one or more sensors, such as one or more cameras 318. For example, one or more cameras 318 may be mounted to the frame 300 and oriented to image the patient's left eye 108 and one or more cameras 318 may be mounted to the frame 300 and oriented to image the patient's right eye 108. Alternatively, a single set of one or more cameras 318 may be repositioned in order to image the right or left eye 108. For example, one or more cameras 318 may be mounted to the staging assembly 312. Where two or more cameras 318 image an individual eye 108, multiple images may be used to identify features in three dimensions.

The cameras 318 may be color or infrared cameras. The cameras 318 are just one example of an imaging device that may be used. Other imaging devices and images obtained from such imaging devices may be used in place of the cameras 318 and images received from the cameras 318. For example, other imaging devices may include an optical coherence tomography (OCT) device, scanning laser ophthalmoscope (SLO), or other type of imaging device. An OCT device is particularly helpful for tracking the location of a needle during insertion, injection, and withdrawal.

The controller 110 may receive images from the one or more cameras 318 and detect features such as an end of the needle of the injection assembly 316 or other recognizable feature of the injection assembly 316 and one or more items of anatomy such as the limbus 320 of the patient's eye 108. Other items of anatomy that may be identified may include the lens and the retina. The controller 110 may then control actuators of the staging assembly 312 such that a needle of the injection assembly 316 will insert within the patient's eye 108 at a prescribed orientation at a point that is a prescribed distance from the limbus 320, such as between 3 and 3.5 millimeters for an aphakic/pseudophakic eye and between 3.5 and 4 millimeters for a phakic eye. In some embodiments, the controller 110 generates a three-dimensional model of the eye 108 and uses the model to precisely guide the needle in order to avoid damaging ocular tissue, such as the lens, retina, or other items of anatomy.

One or more other sensors may be incorporated into the drug delivery assembly 102, such as an intraocular pressure (IOP) sensor 322. The IOP sensor 322 may be a contact or non-contact sensor and may be used during intravitreal injection to ensure that the TOP of the patient's eye 108 does not increase to unsafe levels. There may be separate IOP sensors 322 for each eye or a single IOP sensor 322 may be mounted at different positions on the frame 300 or be mounted to the staging assembly 312 in order to measure the IOP of each eye 108.

The drug delivery assembly 102 may include one or more fixation targets 324. The fixation target 324 may be embodied as a static image, light source, screen for displaying a fixation target, or other device. A separate fixation target 324 may be provided for each eye 108 or a single fixation target may be moved between positions for the left eye 108 and the right eye 108, such as by being mounted to the staging assembly 312. In some embodiments a single fixation target 324 is centrally located to be used for both eyes 108, i.e., patient may direct each eye 108 toward the nose of the patient in order to expose the sclera for receiving an injection. Alternatively, a single screen implementing the fixation target 324 may display a fixation target at a different location for each eye 108. The location of the fixation target may be adjusted using software executed by the controller 110 or by a remote observer in order to induce the patient to position the eye 108 at a desired angle.

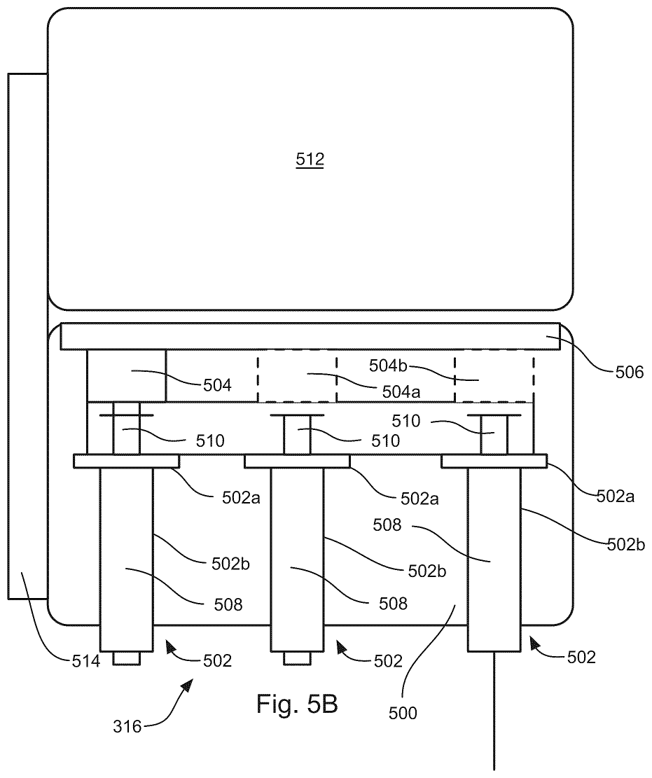

Referring to FIGS. 4A and 4B, the staging assembly 312 may be schematically represented as shown with the understanding that relative dimensions may be different from those shown. The staging assembly 312 may include a base 400 configured to secure to the mounting structure 302 and secure in place with the latch 314. One or more linear actuators 402, 404, 406 may be mounted thereto. For example, linear actuator 402 induces movement in an X direction, linear actuator 404 induces movement in a Y direction, and linear actuator 406 induces movement in the Z direction, where the X, Y, and Z directions are substantially (e.g., within 3 degrees of) perpendicular to one another. In the illustrated embodiment, linear actuator 402 is mounted to the base 400, linear actuator 404 is mounted to linear actuator 402 and is moved by the linear actuator 402 in the X direction, linear actuator 406 is mounted to linear actuator 404 and is moved by linear actuator 404 in the Y direction, and the injection assembly 316 is mounted to the linear actuator 406. The linear actuator 406 may induce motion of the injection assembly 316 toward and away from the eye 108 of the patient up to and possibly including driving a needle of the injection assembly 316 into the eye 108 of the patient. The ranges of motion of the linear actuators 402, 404, 406 need not be equal. For example, the degree of misalignment in the X and Y directions may be relatively small compared to the movement along the Z direction required to move the needle of the injection assembly 316 into proximity to the eye 108 and possibly drive the needle into the eye 108 to a prescribed depth.

The illustrated linear actuators 402, 404, 406 are exemplary only. The arrangement, i.e., stacking, of the linear actuators 402, 404, 406 may be in a different order from that shown. One or both of the actuators 402, 404 may be embodied as rotational actuators having axes of rotation parallel to the X and Y directions, respectively. One or both of the linear actuators 402, 404 may be embodied as arcuate actuators centered on axes parallel to the X and Y directions, respectively, and having a remote center of motion, such as a remote center of motion located at the surface of the patient's eye 108 or offset therefrom.

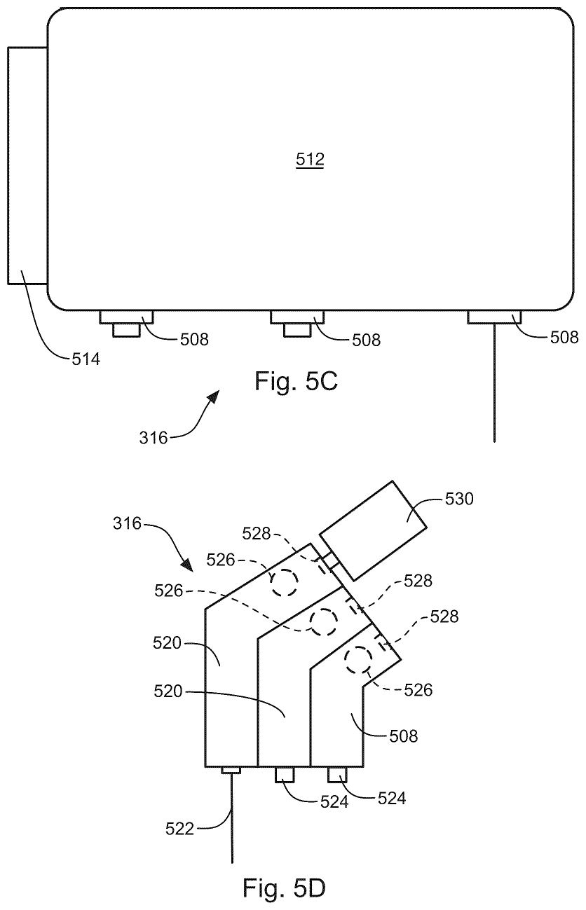

Referring to FIGS. 5A to 5C, the injection assembly 316 may have some or all of the attributes and/or functionalities described below. The injection assembly 316 may include a tray 500 defining one or more recesses 502 for receiving syringes, such as three recesses 502 for receiving syringes containing an anesthetic, a disinfectant, and a drug to be delivered by intravitreal injection. For example, each recess 502 may include a groove 502a for receiving a flange of a syringe and a recess 502b connected to the groove 502a for receiving the barrel of the syringe.

A plunger actuator 504 is positioned to depress the plunger 510 of syringes 508 positioned within the recesses 502. In some embodiment, a single plunger actuator 504 is used and is moved by a positioning actuator 506 between the illustrated position and two other positions 504a, 504b in order to depress the plunger 510 of syringes positioned in each of the recesses 502. In other embodiments, a separate plunger actuator 504 is provided to depress the plunger 510 of a syringe 508 positioned in each recess 502.

Syringes 508 may be retained within the recesses 502 by means of a lid 512 or other retention structure. The lid 512 may be coupled to a lid actuator 514 that can be moved into the open position of FIGS. 5A and 5B and into the closed position of FIG. 5C in which the lid 512 is positioned over the syringes 508 positioned within the recesses 502. The lid 512 may be flat or may include recesses that receive portions of the syringes 508 when the lid 512 is in the closed position over the tray 500. For example, the lid 512 may include recesses 502 similarly to the tray 500, each recess 502 including a groove 502a for receiving a flange of a syringe 508 and a recess 502b for receiving the barrel of a syringe 508.

FIG. 5D illustrates an alternative implementation for the injection assembly 316. In the illustrated embodiment, the injection assembly 316 includes one or more reservoirs 520, such as the illustrated reservoirs 520 for containing a drug to be administered, an anesthetic, and a disinfectant. The reservoirs 520 may be separate members or joined together by fasteners, placement in a common housing, or co-molding. Each reservoir 520 may have an outlet formed thereon or secured thereto, such as in the form of a hypodermic needle 522 or nozzle 524 for dispensing fluid.

Each reservoir 520 may have a pump 526 associated therewith. The pump 526 of each reservoir 520 may be used to force fluid out of the outlet of the reservoir 520. The pump 526 may be replaced with other propulsion sources. For example, pressurized fluid may be forced into a reservoir 520 and engage a piston or bladder in order to force fluid out of the reservoir 520.

Each reservoir 520 may have an inlet 528 for filling the reservoir 520. The inlet 528 may be coupled to a vial 530 or syringe containing fluid to be loaded into the reservoir 520. The fluid may be forced into the reservoir 520 using a syringe or other pressure source. Alternatively, the pump 526 of a reservoir 520 may be activated in order to draw fluid out of a vial 530 through the inlet 528 of the reservoir 520. In other implementations, fluid may be drawn through the inlet 528 or outlet of a reservoir 520 and into a bladder within a reservoir 520 by reducing pressure in the reservoir 520 around the bladder, such as through a port for coupling to a pneumatic pressure source. In some embodiments, the reservoirs 520 may be large enough to store multiple doses. In such embodiments, the injection assembly 316 may include refrigeration to avoid degradation of a drug to be injected.

The inlet 528 may include a one-way valve, self-sealing polymer defining a hole for receiving a needle, removable cap, or other closure mechanism. In some embodiments, the injection assembly 316 is a disposable cartridge that is pre-loaded with fluid such that an inlet 528 is omitted. For example, the reservoirs 520 may be filled through the outlet thereof at the time of manufacture.

The injection assembly 316 according to the embodiments of FIGS. 5A to 5C and FIG. 5D may be coupled to the controller 110 and a power source for powering the actuators 504, 506, 514 or pumps 526 by means of contacts on the injection assembly that engage corresponding contacts on the staging assembly 312, a socket into which a connector coupled to the controller 10 is plugged, or some other connection.

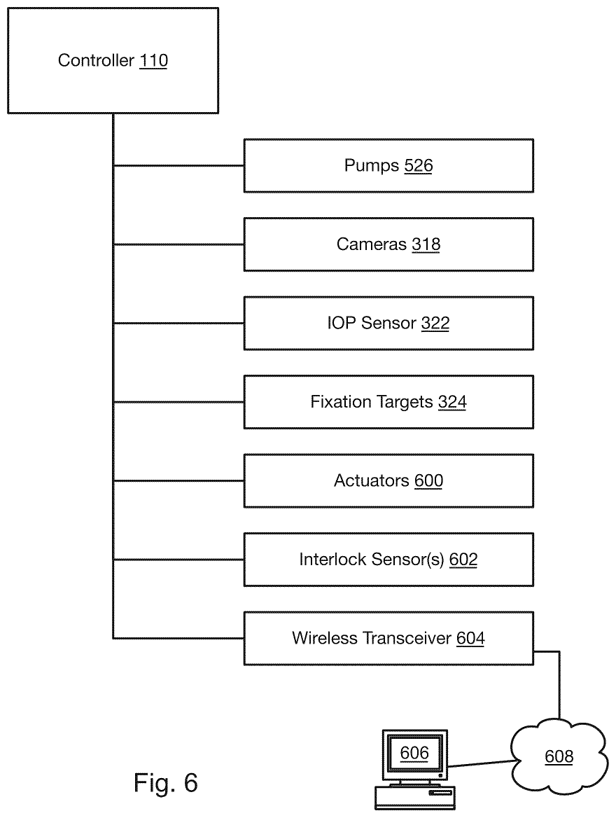

Referring to FIG. 6, the controller 110 is coupled to some or all of the one or more pumps 526, the one or more cameras 318, the one or more IOP sensors 322, and the one or more fixation targets 324 by means of wired or wireless connections, optical fibers, or other type of connection. The controller 110 is configured to activate and deactivate the one or more pumps 526, one or more cameras 318, and one or more IOP sensors 322, and one or more fixation targets 324. The controller 110 is configured to receive images from the one or more cameras 318 and IOP readings from the IOP sensor 322. The controller 110 may receive feedback from the one or more pumps 526, such as measurements of pressure at the input and/or output of each pump of the one or more pumps 526, current drawn by each pump of the one or more pumps 526, or other information. In some embodiments, feedback from the one or more pumps 526 may be used to obtain an estimated IOP reading and the one or more IOP sensors 322 may be omitted.

The controller 110 may be coupled to one or more other components, such as actuators 600 including some or all of the actuators 402, 404, 406, 504, 506, 514 described herein in order to control activation of the actuators 600 and possibly receive feedback regarding the state of some or all of the actuators 600 (e.g., current angular or translational position, velocity, and/or acceleration). The staging assembly 312, mounting structures 302, and injection assembly 316 are detachable from one another and may include electrical contacts to conduct electrical power and control signals from the controller 110 to the actuators 504, 506 or pumps 526 of the injection assembly 316.

The controller 110 may be coupled to one or more interlock sensors 602 that detect a state of the drug delivery device 100 relative to the head 106 of the patient. For example, interlock sensors 602 may sense whether a patient's chin is in contact with a chin rest, whether a patient's forehead is in contact with a forehead rest, whether the staging assembly 312 is properly mounted to one of the mounting structures 302, whether the injection assembly 316 is properly mounted to the staging assembly 312, or that any of the components described herein is positioned and functioning properly.

The controller 110 may be coupled to a wireless transceiver 604. Although the drug delivery device 100 is designed and able to be used by a patient in the patient's own home, medical supervision and control may still be provided remotely. Accordingly, the operation of the controller 110 may be subject to authorization and instructions received from a computing device 606 over a network 608 by way of the wireless transceiver 604. The controller 110 may authenticate a remote observer using the computing device 606 prior to permitting control using the computing device 606. The remote observer may additionally interact with the patient during a procedure, such as by means of an output device such as a screen, speakers, or other device incorporated into the drug delivery device 102. Instructions to the patient may be output from the output device either automatically or in response to instructions from the remote observer. The patient may interact with the remote observer using an input device incorporated into the drug delivery device 102, such as the one or more cameras 318, a microphone, a touch screen, pointing device, a keyboard, or other input device.

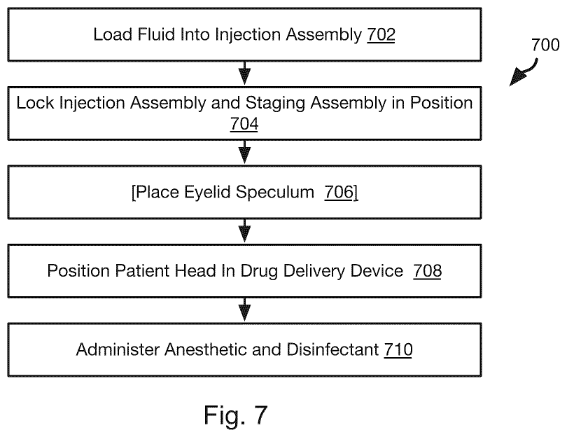

FIG. 7 is a process flow diagram of a method 700 for preparing for drug delivery using the drug delivery device 100. The method 700 includes loading, at step 702, fluid into the injection assembly 316, including the drug to be delivered, an anesthetic, and a disinfectant as described above with respect to FIGS. 5A to 5D. As an alternative, a disposable injection assembly 316 may be provided that is already loaded with fluid such that step 702 is not performed by the patient.

The method 700 includes locking, at step 704, the injection assembly 312 into place on the staging assembly 312. Before or after locking the injection assembly 312 into place on the staging assembly 312, the staging assembly 312 may also be locked, at step 704, into place to one of the mounting structures 302. However, where two staging assemblies 312 are used or the staging assembly 312 is already locked in place in the correct mounting structure 302, step 704 may omit locking of the staging assembly 312 into place.

In some embodiments, the patient may place, at step 706, a speculum in the eye 108 to be treated in order to move the eyelid out of the way. In other embodiments, the patient is relied upon to maintain the eyelid out of the way such that a speculum is not used. In still other embodiments, an actuated speculum is incorporated into the staging assembly 312 and withdraws the eyelid automatically.

The method 700 includes positioning, at step 708, the patient's head 106 into the drug delivery device 100. Step 708 may include placing the patient's chin on the chin rest 104 and the patient's forehead and eyes into the drug delivery assembly 102 and possibly engaging a headband or clamp to restrict movement of the patient's head 106 relative to the drug delivery assembly 102. Step 708 may include placing the patient's head 106 into the helmet 200 and possibly tightening a headband 202 using a tensioner 204.

The method 700 may include administering, at step 710, an anesthetic and a disinfectant. Step 710 may be an automated step in which each of the anesthetic and disinfectant is dispensed by depressing a plunger of a syringe using a plunger actuator 504 or activating a pump 526. The outlets of the syringes 508 or reservoirs 520 used to dispense the anesthetic and disinfectant may be placed close to the eye 108 being treated, e.g., within 1 millimeter, or in contact with the eye 108. Alternatively, fluid may be sprayed at step 710 such that such proximity is not required. In some embodiments, step 710 is performed manually by a patient prior to performing step 708.

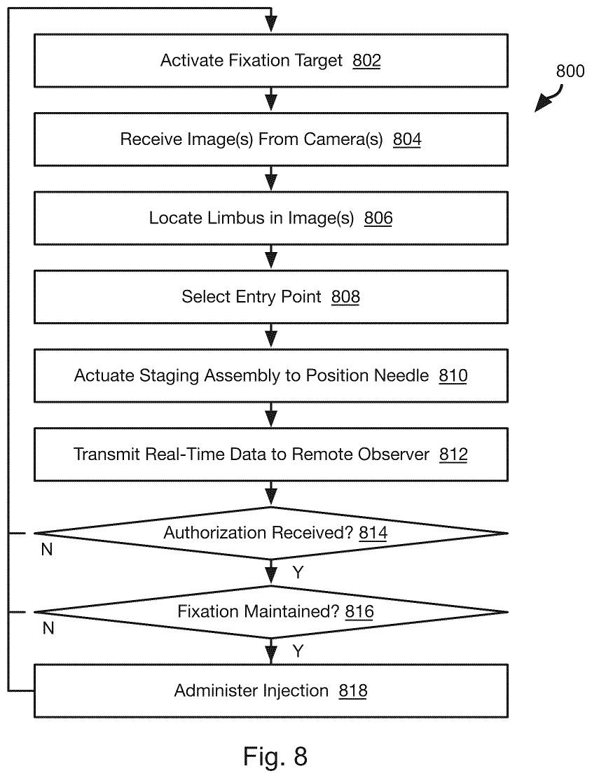

FIG. 8 is a process flow diagram of a method 800 for administering an intravitreal injection using the drug delivery device 100. The method 800 may be performed following performance of the method 700 by the controller 110 activating components of the drug delivery device 100. The method 800 may be performed after waiting for a prescribed time following step 710 of the method 700 for anesthetic and disinfectant to function.

The method 800 includes activating, at step 802, a fixation target 324. Activating the fixation target 324 may include activating a light, e.g., light emitting diode, displaying an image on the screen, or otherwise providing a visual indicator that is visible to the eye to be treated. Where the fixation target is a static visible structure, step 802 may be omitted. Step 802 may include outputting visual or audible instructions to the patient to fixate on the fixation target 324.

The method 800 includes receiving, at step 804, one or more images from the one or more cameras 318 having the eye 108 to be treated in the field of view thereof. The images received at step 804 may be received in the form of one or more video feeds from the one or more cameras 318.

The method 800 includes locating, at step 806, the limbus 320 represented in the one or more images. Step 806 may be performed by registering one or more labeled reference images with respect to the one or more images, the labeled reference image including a label of the limbus 320. Step 806 may be performed using a machine learning model trained to perform the task, machine vision algorithm, or other approach.

The method 800 may include selecting, at step 808, an entry point relative to the limbus 320. For example, any point within a band of permitted offsets from the limbus 320, such as between 3 and 3.5 millimeters for an aphakic eye and between 3.5 and 4 millimeters for a phakic eye. The angular position of the entry point about the optical axis of the eye 108 to be treated may be selected as a position that is not obscured by an eyelid of the patient.

The method 800 may include actuating, at step 810, the staging assembly 312 such that the needle of the injection assembly 316 is pointed at the entry point along the actuation direction of the actuator 406. In some embodiments, only translational positioning is performed. However, in others, step 810 may include changing an orientation of the injection assembly 316 such that a needle of the injection assembly 316 is oriented at a desired angle relative to the normal vector of the selected entry point. The desired angle is as known in the art of intravitreal injections and may be selected such that upon insertion of the needle, the needle avoids contact with the lens and retina while placing medication near the retina or area of the retina to be treated. Note that in some applications, the needle will be relatively short (e.g., about 8 mm) such that the angle and depth are not critical for avoiding harm to ocular tissue. In other applications, the needle is used to provide a sub-retinal injection such that angle and depth of penetration are important. In some embodiments, if the range of motion of the staging assembly 312 is not sufficient to position the needle pointed at the selected entry point, the method 800 may end or the user may be instructed how to adjust the patient's head 106 relative to the drug delivery assembly 102 to make proper positioning possible.

Step 810 may be performed along with one or more additional iterations of some or all of steps 804, 806, 808 to account for movement of the eye 108 to be treated. Likewise, step 810 may include identifying a representation of the needle in the one or more images received from the one or more cameras 318 and using the representation as feedback to guide positioning of the needle relative to the selected entry point.

The method 800 may include transmitting, at step 812, real time data to a remote observer, such as to the computing device 606 of an authenticated medical professional. The real time data may include images from the one or more cameras 318, such as by forwarding a video feed from the one or more cameras 318. The real time data may include a representation of the selected entry point from step 808 and a location and orientation of the needle, such as in the form of annotations to images from the one or more cameras 318. The real time data may include reports of successful application of anesthetic and disinfectant, which may include an amount of each applied. The real time data may include outputs of one or more interlock sensors 602 indicating whether the patient is properly positioned, and components of the drug delivery device are locked in place and functioning correctly.

The method 800 may include performing one or more verifications prior to administering, at step 818, an intravitreal injection. In some embodiments, some or all of steps 802-812 may be repeated until the verifications are successful or the method 800 is ended by the patient or an observer. The verifications may include verifying, at step 814, that authorization was received from the remote observer and verifying, at step 816, that fixation of the eye 108 to be treated has been maintained. For example, step 816 may include verifying, using a video feed from the one or more cameras 318, that movement of the eye 108 to be treated is below a maximum threshold, e.g., less than 1 degree, 0.5 degrees, or 0.1 degrees. Step 816 may include verifying fixation of the eye 108 to be treated (e.g., movement less than the maximum threshold) for a minimum period of time, such as from 1 to 3 seconds. Other verifications may include verifying the identity of the patient, such as by verifying that an iris or retina in one or more images from the one or more cameras 318 matches one or more reference images of an iris and/or retina or representation thereof accessed by the controller. In some embodiments, an explicit instruction must be received from the patient to verify that step 818 can be performed, such as in the form of pressing or releasing a button, a verbal command, or visible gesture detected by a camera coupled to the controller 110.

Administering the intravitreal injection at step 818 may include activating the actuator 406 to drive a needle into the eye 108 to be treated and activating a plunger actuator 504 or pump 526 to force fluid through the needle and into the eye 108. Step 818 may be performed simultaneously with one or more actions that may include verifying continued authorization by the remote observer. For example, a remote observer may continue to receive a video feed from the one or more cameras 318. The remote observer may hold a button throughout the procedure and release the button in the event that the remote observer believes that the intravitreal injection should be aborted. In response to receiving notification of release of the button, the controller 110 may abort the intravitreal injection. This approach to continued authorization is exemplary only and other approaches may be used, such as the remote observer pressing a button or interacting with another user interface element to invoke transmission of an instruction to the controller 110 to abort the intravitreal injection by the controller 110. Step 818 may likewise be aborted in response to an input from the patient in the form of pressing or releasing a button, a verbal command, or visible gesture detected by a camera coupled to the controller 110.

Likewise, fixation may continue to be evaluated as described above with respect to step 816. In the event that fixation is not maintained, the intravitreal injection may be aborted. The IOP within the eye 108 to be treated may be evaluated using outputs of the IOP sensor 322. In the event that the IOP rises faster than a prescribed rate or above a prescribed pressure, the intravitreal injection may be aborted, or the rate of drug delivery may be slowed. In some embodiments, the rate of injection of fluid is regulated based on feedback regarding IOP in order to maintain pressure within the eye 108 below a threshold or a pressure-vs-time curve, with time being measured from when fluid injection began.

In some embodiments, the staging assembly 312 may be activated during step 818 in order to at least partially compensate for movement of the eye 108 to be treated relative to the needle. For example, the staging assembly 312 may include one or more strain sensors sensing strain on the needle in one or more dimensions. The controller 110 may activate one or more of the linear actuators 402, 404, 406 to reduce the amount of strain sensed by the strain sensors. Step 818 may be aborted in response to movement of the eye 108 to be treated exceeding the range of motion and/or speed of movement required for the staging assembly 312 to compensate for the movement of the eye 106.

Aborting the intravitreal injection may include causing the actuator 406 to rapidly withdraw the needle of the injection assembly 316 from the eye 108 to be treated to a safe distance from the eye 108 to be treated. Once aborted, the controller 110 may require repetition of the method 700 and 800. Alternatively, once aborted, the controller 110 may disable further intravitreal injections and require the patient to visit a medical professional for the intravitreal injection.

Step 818 may include monitoring the amount of drug delivered, e.g., amount by which a plunger of a syringe was depressed, or amount of pumping performed by the pump 526. Accordingly, an amount of drug that remains to be administered may be determined by the controller 110 and provided to a remote observer or used by the controller 110 to control the amount of drug delivered in a subsequent iteration of the method 800.

Once the methods 700 and 800 are performed for one eye 108 to be treated, the methods 700 and 800 may be repeated for the patient's other eye 108. As described above, one or more components may be transferred, such as transferring the staging assembly 312 manually or automatically to a different mounting structure 302 and fastening the staging assembly 312 to the different mounting structure 302. As also noted above, separate staging assemblies 312 may be used. Accordingly, injection assemblies 316 may be secured to each staging assembly 312 and the methods 700 and 800 may be performed for each eye 108 of the patient in series, in parallel, or in an interleaved manner. For example, administration of disinfectant and anesthetic may be performed for both eyes 108 in parallel whereas fixation and injection (e.g., steps 814-818) may be performed in series.

The methods 700 and 800 are exemplary only and may be modified to perform additional steps or ophthalmic treatments. For example, although a drug to be delivered, an anesthetic, and a disinfectant are mentioned above, other fluids may also be used to treat the eye 108 either before or after an injection. For example, some or all of a cooling spray (e.g., saline), anti-inflammation cream or spray, anti-bleeding solution may also be loaded into the injection assembly 316 and applied using the injection assembly 316. In some embodiments, the staging assembly 312 may include one or more actuators that are activated by the controller to press a pad (e.g., cotton or other absorbent material) against an injection site following injection in order to reduce bleeding. Likewise, an actuated pad incorporated into the staging assembly 312 may be pressed against the eye 108 during injection to resist movement of the eye.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented, or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

1. A drug delivery device comprising:
a drug delivery assembly configured to receive a portion of a head of a patient and comprising:
one or more imaging devices configured to have an eye of the patient in a field of view thereof;
an injection assembly configured to receive a drug to be injected into the eye of the patient; and
a staging assembly including one or more actuators and configured to position the injection assembly relative to the eye of the patient; and
a controller coupled to the one or more imaging devices and the staging assembly, the controller configured to:
receive one or more images from the one or more imaging devices;
detect a location of a limbus of the eye of the patient in the one or more images; and
activate the one or more actuators to drive a needle mounted to the injection assembly into a placement location on the eye of the patient according to the location of the limbus.

2. The drug delivery device of claim 1, wherein controller is coupled to the injection assembly and the controller is further configured to activate the injection assembly to force fluid through the needle into the eye of the patient.

3. The drug delivery device of claim 2, wherein the injection assembly comprises a pump.

4. The drug delivery device of claim 2, wherein the injection assembly is configured to depress a plunger of a syringe positioned within the injection assembly.

5. The drug delivery device of claim 1, wherein the controller is further configured to activate the one or more actuators to drive the needle into the placement location in response to an authorization received from a remote operator over a network.

6. The drug delivery device of claim 1, wherein the controller is further configured to activate the one or more actuators to drive the needle into the placement location in response to verifying fixation of the eye of the patient.

7. The drug delivery device of claim 6, further comprising a fixation target configured to be focused on by the eye of the patient.

8. The drug delivery device of claim 1, wherein the drug delivery assembly comprises first and second mounting structures, the staging assembly configured to mount to either of the first and second mounting structures.

9. The drug delivery device of claim 1, wherein the staging assembly includes only linear actuators.

10. The drug delivery device of claim 1, wherein the drug delivery assembly is mounted to one of (a) a support configured to rest on a support surface and (b) a helmet configured to be worn on the head of the patient.

11. A method for drug delivery comprising:
positioning a portion of a head of a patient in a drug delivery assembly comprising:
one or more imaging devices configured to have an eye of the patient in a field of view thereof;
an injection assembly configured to receive a drug to be injected into the eye of the patient; and
a staging assembly including one or more actuators and configured to position the injection assembly relative to the eye of the patient;
receiving, by a controller, one or more images from the one or more imaging devices;
detecting, by the controller, a location of a limbus of the eye of the patient in the one or more images; and
activating, by the controller, the one or more actuators to drive a needle mounted to the injection assembly into a placement location on the eye of the patient according to the location of the limbus.

12. The method of claim 11, further comprising activating, by the controller, the injection assembly to force fluid through the needle into the eye of the patient.

13. The method of claim 12, wherein activating the injection assembly comprises activating a pump of the injection assembly.

14. The method of claim 12, wherein activating the injection assembly comprises depressing a plunger of a syringe positioned within the injection assembly.

15. The method of claim 11, further comprising activating, by the controller, the one or more actuators to drive the needle into the placement location in response to an authorization received from a remote operator over a network.

16. The method of claim 11, further comprising activating, by the controller, the one or more actuators to drive the needle into the placement location in response to verifying fixation of the eye of the patient.

17. The method of claim 16, further comprising activating, by the controller, a fixation target configured to be focused on by the eye of the patient.

18. The method of claim 11, further comprising:
mounting the staging assembly to a first mounting structure of the drug delivery assembly; and
mounting the staging assembly to a second mounting structure of the drug delivery assembly.

19. The method of claim 11, wherein the staging assembly includes only linear actuators.

20. The method of claim 11, wherein the drug delivery assembly is mounted to one of (a) a support configured to rest on a support surface and (b) a helmet configured to be worn on the head of the patient.
